SEQUENCE LISTING (17 sequences)

SEQ ID NO: 1 - DNA, 9 bp, SARS-CoV-2
atctgtcaa

SEQ ID NO: 2 - RNA, 9 nt, SARS-CoV-2
uugacagau

SEQ ID NO: 3 - DNA, 20 bp, Artificial Sequence
  disDNA sequence
tcaagcagcagcaaagcaag

SEQ ID NO: 4 - DNA, 9 bp, Artificial Sequence
  P2p primer
gtcaagcag

SEQ ID NO: 5 - DNA, 9 bp, Artificial Sequence
  P2* primer
gttcaatct

SEQ ID NO: 6 - DNA, 8 bp, Artificial Sequence
  P1c* primer
ctgattga

SEQ ID NO: 7 - DNA, 9 bp, Artificial Sequence
  P1 (csp) primer
agattgaac

SEQ ID NO: 8 - DNA, 18 bp, Artificial Sequence
  Reporter oligo
ctgcttgacagattgaac

SEQ ID NO: 9 - DNA, 11 bp, Artificial Sequence
  Quencher oligo
ctgtcaagcag

SEQ ID NO: 10 - RNA, 40 nt, Artificial Sequence
  RNA template
cuugcuuugcugcugcuugacagauugaaccagcuugaga

SEQ ID NO: 11 - DNA, 18 bp, Artificial Sequence
  LIDA ligation product
gttcaatctgtcaagcag

SEQ ID NO: 12 - protein, 73 aa, SARS-CoV-2
MLQSCYNFLKEQHCQKASTQKGAEAAVKPLLVPHHVVATVQEIQLQAAVGELLLLEWLAMAVMLLLLCCCLTD

SEQ ID NO: 13 - protein, 70 aa, Human-CoV
MLPPCYNFLKEQHCQKASTQREAEAAVKPLLAPHHVVAVIQEIQLLAAVGEILLLAWLAEVVKLPSRYCC

SEQ ID NO: 14 - protein, 70 aa, Bat-CoV
MLPSCYNFLKEQHCQKASTQRGAEVAVNLHLAPHHVVAVIQEIQLLAAVGEVLLLDWLAEVVKLPSRYCC

SEQ ID NO: 15 - DNA, 170 bp, SARS-CoV-2
tcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtca

SEQ ID NO: 16 - protein, 56 aa, SARS-CoV-2
VATVQEIQLQAAVGELLLLEWLAMAVMLLLLCCCLTDTSLRAKCLVKANNNKAKLS

SEQ ID NO: 17 - DNA, 170 bp, SARS-CoV-2
agcgttgtcaagttctttaagttgaggtccgtcgtcatccccttgaagaggacgatcttaccgaccgttaccgccactacgacgagaacgaaacgacgacgaactgtctaacttggtcgaactctcgttttacagaccatttccggttgttgttgttccggtttgacagt

(19) United States
(12) Patent Application Publication
(10) Pub. No.: US 2024/0279753 A1
(43) Pub. Date: Aug. 22, 2024

(54) ASSAYS FOR SARS-COV-2 BY LESION INDUCED DNA AMPLIFICATION (LIDA)

(71) Applicant: READYGO DIAGNOSTICS LIMITED, Wiltshire (GB)

(72) Inventor: Benjamin David Cobb, North Wraxall (GB)

(21) Appl. No.: 18/571,719

(22) PCT Filed: Jun. 20, 2022
(86) PCT No.: PCT/EP2022/066736

§ 371 Date: Dec. 19, 2023

(30) Foreign Application Priority Data
Jun. 21, 2021 (GB) 2108853.9

(51) Int. Cl. C12Q 1/70

(52) U.S. Cl. CPC C12Q 1/701

(57) ABSTRACT

Assays for SARS-CoV-2 are described, together with lesion induced DNA amplification (LIDA)-based methods for amplifying RNA or DNA.

### FIELD OF THE INVENTION

The present invention relates to a nucleic acid assay and a method for amplifying or amplifying and detecting nucleic acids. In particular embodiments, the invention relates to an assay for detecting SARS-CoV-2 nucleic acids.

### BACKGROUND TO THE INVENTION

The SARS-CoV-2 pandemic has led to development of a number of assays for detecting the virus. Two of the most common are antigen-based lateral flow assays, which detect presence of specific viral proteins, using conjugated antibodies to bind spike, envelope, membrane or nucleocapsid proteins; or RT-PCR assays, which amplify specific viral genome sequences to detect presence of the virus. Each of these has advantages and disadvantages; lateral flow assays are rapid, giving results in as little as 30 minutes, but relatively less sensitive and prone to false negatives. RT-PCR tests are more accurate, but are not seen as a rapid option given the need generally to return samples to a laboratory for testing. It would be beneficial to have alternative testing options available. Further, given rates of viral mutation in the genes and proteins normally assayed, there may be reductions in sensitivity of these assays. It would therefore be beneficial to adopt a different region of the viral genome in the assay.

Various isothermal amplification methods are known for amplification of nucleic acids which may be suitable for incorporating into an alternative assay for SARS-CoV-2. These include NASBA (nucleic acid sequence-based amplification); LAMP (loop-mediated isothermal amplification); HAD (helicase-dependent amplification); RCA (rolling circle amplification); MDA (multiple displacement amplification); WGA (whole genome amplification, including MALBAC, LIANTI, DOP-PCR); and RPA (recombinase polymerase amplification). The benefit of isothermal amplification is that it does not require thermal cycling to amplify the target, and so may not require specific equipment to carry out the assay.

A further isothermal technique is RT-LIDA (reverse transcription lesion-induced DNA amplification). The LIDA technique is described generally in U.S. Pat. No. 9,193,993, and is a method for isothermally amplifying a DNA sequence involving hybridizing a destabilizing DNA template to complementary nucleotide fragments to form a first nicked duplex; ligating the first nicked duplex to form a product duplex comprising the DNA sequence and the template, wherein the product duplex is capable of dissociating to release the DNA sequence and the template; and repeating these steps to generate multiple copies of the template and the DNA sequence. Where the initial template is RNA, then an initial step is also included to generate cDNA from the RNA. RT-LIDA is also described in Alladin-Mustan et al, “Reverse transcription lesion-induced DNA amplification: An instrument-free isothermal method to detect RNA”; Analytica Chimica Acta, Volume 1149, 2021, 238130, https://doi.org/10.1016/j.aca.2020.12.005.

In developing the SARS-CoV-2 assay described herein, the present inventor has further developed a modification of RT-LIDA which may provide improved amplification results. In particular, in conventional RT-LIDA, self-ligation of primers can be an issue, resulting in false positives. It is believed that the modified assay presented here reduces the occurrence of such false positives, among other advantages.

Although the specific assay described herein is an RT-LIDA amplification of a portion of the SARS-CoV-2 genome, it will be understood that a) the same portion of SARS-CoV-2 genome may be detected using other methods, in particular isothermal amplification; and that b) the modified RT-LIDA described herein is of more general applicability than for this assay. A key benefit of the assay described herein is that it allows for isothermal amplification and detection in a single reaction vessel, and can be performed with no specialised equipment beyond the assay reagents, if desired.

### SUMMARY OF THE INVENTION

According to an aspect of the present invention, there is provided an assay for SARS-CoV-2 wherein a portion of a nucleic acid coding for a Leu-Thr-Asp (LTD) sequence at or near the terminus of the ORF9c protein is amplified and detected.

Also provided is a method for detecting SARS-CoV-2 in a sample, the method comprising: generating cDNA from an RNA present in the sample; amplifying a portion of the cDNA using an amplification process specific for a portion of the cDNA corresponding to the SARS-CoV-2 genome coding for ORF9c; and detecting the presence of a portion of amplified cDNA coding for a Leu-Thr-Asp (LTD) sequence at or near the terminus of the ORF9c protein.

The SARS-CoV-2 genome includes a gene coding for ORF9c (previously known as ORF14). This is a 70 amino acid protein which was previously of unknown function and present in Human SARS and Bat CoV. In SARS-CoV-2 the ORF9c protein is 73 amino acids long and has a 9 bp insert coding 3 additional amino acids (LTD) at the terminus of the transcript. A comparison of the ORF9c amino acid sequences from SARS-CoV-2 (SEQ ID NO: 12), Human SARS (SEQ ID NO: 13), and Bat CoV (SEQ ID NO: 14) is given in FIG. 2. More recently this protein has been shown to be play a critical and important role in the viruses' ability to evade the human immune system. The LTD insert appears to be highly conserved in SARS-CoV-2, making it a suitable target for detection. In particular, although the SARS-CoV-2 genome has well distributed mutations recorded across the genome, there are minimal mutations found in ORF9c and even fewer recorded in this LTD insertion. Given the possible role of ORF9c in viral infectivity, this lack of variability suggests a selective advantage for the virus, and hence the insertion is useful as a diagnostic.

The cDNA sequence encoding the LTD insertion is AAC TGT CTA (SEQ ID NO: 1). The genomic sequence will of course be the corresponding RNA sequence (UUG ACA GAU, SEQ ID NO: 2). Amplification may be of a DNA sequence spanning this insertion, and detection via a probe which binds a sequence comprising this insertion.

In preferred embodiments, amplification is via RT-LIDA. LIDA is a simple-to-implement amplification technique based on a modification of the Ligase Chain Reaction (LCR). It operates at room-temperatures between 18° C. to 37° C. to provide rapid (≤20 minutes) amplification of a selected target. It uses four oligonucleotide primers and a single enzyme making it significantly less complex compared to other isothermal chemistries.

Also provided is a method of amplifying a target RNA molecule in a sample, the method comprising:
a) providing a sample containing said target RNA molecule;
b) providing first and second DNA primers (P2p, P2*) complementary to contiguous portions of said target RNA molecule;
c) providing third and fourth DNA primers (P1c*, P1(csp)) complementary to the P2p and P2* primers, wherein the third and fourth DNA primers are destabilising primers;
d) providing a displacement DNA strand (disDNA) overlapping with the P2p and or the P2* primers and complementary to the target RNA molecule;
e) allowing the P2p and P2* primers to anneal to the target RNA, to form a RNA-nicked DNA duplex;
f) ligating the P2p and P2* primers, to form an RNA-DNA duplex, wherein the DNA strand is ligated P2p-P2*;
g) allowing the disDNA to displace the ligated P2p-P2* DNA strand from the duplex;
h) allowing the destabilising primers to anneal to the ligated P2p-P2* DNA strand to form a DNA-nicked DNA duplex;
i) ligating the P1c* and P1(csp) primers, to form a product DNA duplex having a first cDNA strand corresponding to the P2p-P2* primers, and a second destabilised cDNA strand corresponding to the destabilising P1c*-P1(csp) primers;
j) allowing the destabilised cDNA strand to dissociate from the first cDNA strand;
k) allowing the P2p and P2* primers to anneal to the destabilised cDNA strand, and allowing the destabilising primers to anneal to the first cDNA strand to form DNA-nicked DNA duplexes;
l) ligating the primers to form product DNA duplexes having a first cDNA strand corresponding to the P2p-P2* primers, and a second destabilised cDNA strand corresponding to the destabilising P1c*-P1(csp) primers; and
m) repeating steps j) to l) to produce multiple copies of the first and second cDNA strands;
   wherein the ligating steps are carried out with a DNA ligase having no single base overhang or blunt end ligating ability.

As discussed herein, the inventor has determined that use of a DNA ligase having no single base overhang or blunt end ligating ability provides for enhanced accuracy and reduced background from the method. In preferred embodiments, the ligase is PBCV-1 DNA ligase, although others may be used, including engineered ligases which have been modified from the natural form to reduce or remove a single base overhang or blunt end ligating ability. The reduction of background amplification from use of such a ligase means that components can be included in the reaction mix which accelerate the ligase reaction (for example, crowding agents, such as PEG). With conventional ligases having single base overhang ligating ability, such as T4 ligase, these have to be omitted because they promote the co-amplification of this background.

The destabilising primers may include one or more features selected from the presence of an abasic site or a mismatch with the corresponding complementary sequence. In embodiments, one primer includes a mismatch, and one primer includes an abasic site. Preferably the P1c* primer includes a mismatch; this may be an A:T mismatch (that is, a perfectly complementary sequence may include a G or C, whereas the mismatch includes an A or a T). Preferably the mismatch is internal to the primer; that is, at least 2, 3, 4, or more nucleotides distant from the 5′ and the 3′ end. In embodiments, the P1(csp) primer includes an abasic site. The abasic site is preferably at an end of the primer, preferably the 5′ end.

The primers are designed so as to hybridise to contiguous portions of the target. To allow for ligation, the upstream primer of each pair (that is, the primer which hybridises to a region on the target 5′-wards of the other primer) includes a phosphate group at the 5′ end. For example, the P2p and P1(csp) primers may include this phosphate group.

The method may further comprise the step of detecting at least one of the cDNA strands. Preferably at least one of the primers includes a label. For example, the P2* primer may include a label. In embodiments, one primer from each pair includes a detectable label (eg, the P2* primer and the P1c* primer). This allows detection of both cDNA strands. The label may be a fluorescent label, for example, a fluorescein group.

The detection step may comprise capturing at least one of the cDNA strands via a complementary oligonucleotide (Ro, a reporter oligonucleotide) immobilised on a solid support. Where the cDNA strand is labelled, this can be used to localise the label to a specific location; for example, in order to develop a line or other indicator to show detection of the target sequence.

In an embodiment, the immobilised complementary oligonucleotide Ro may initially be hybridised to a partially-complementary oligonucleotide (for example, an oligonucleotide having one or more mismatches, or an oligonucleotide which is shorter than the immobilised oligonucleotide); and capturing the cDNA strand comprises 35 allowing the cDNA strand to displace the partially-complementary oligonucleotide. It will be apparent that the partially-complementary oligonucleotide is partially identical to the cDNA. In a preferred embodiment the partially-complementary oligonucleotide is shorter than the immobilised oligonucleotide and is shorter than the cDNA. The relative lengths are preferably selected such that, in the event that displaced oligonucleotide hybridises to free cDNA or primers, the ligase is unable to activate. This reduces false positives and further allows the detection step to take place in the same environment as the amplification step (that is, in the presence of the primers). In embodiments, the immobilised complementary oligonucleotide Ro and partially-complementary oligonucleotide (Qo) include a reporter-quencher pair (for example, Ro may include a reporter and Qo includes a quencher). Displacement of the Qo oligonucleotide by the cDNA strand separates the reporter-quencher pair and allows the reporter to be detected. This approach avoids the need to include a label in the initial primers. Any compatible reporter-quencher pair may be used; for example FAM or VIC for the reporter dye and TAMRA for the quencher dye. In other embodiments, the oligonucleotides Ro and Qo may be linked as a single molecule, either through an intervening section of nucleic acid, or via a non-nucleic acid linker. In further embodiments, where a reporter-quencher pair is used, the Ro oligonucleotide need not be immobilised on a solid support, but may be in solution. In an example, the Ro and Qo oligonucleotides in solution may be joined by a linker (nucleic acid or non-nucleic acid).

In some embodiments, at least one of the primer sequences includes a tag (for example, a nucleic acid tag) which is not part of the target sequence to be amplified. This tag may be used in the detection or other steps of the method, as described herein.

In embodiments, the primer and other sequences are as follows:

SEQ ID NO: 3
3′-GAA CGA AAC GAC GAC GAA CT-5′,-disDNA sequence
SEQ ID NO: 4
3′-GAC GAA CTGp-5′,-P2p seq
SEQ ID NO: 5
3′-TCT AAC TTG-5′,-P2* seq,
SEQ ID NO: 6
5′-CTG ATT GA-3′,-P1c* seq
SEQ ID NO: 7
5′-p(Ab) AGA TTG AAC-3′,-P1(csp) seq,
(Ab) is abasic site.

In further embodiments, the Ro and Qo oligonucleotide sequences are:

SEQ ID NO: 8
5′-CTG CTT GAC AGA TTG AAC-3′, reporter oligo
SEQ ID NO: 9
3′-GAC GAA CTG TC-5′,, quencher oligo.

It is also possible to use a similar method to amplify DNA, with the addition of a DNA unfolding enzyme (eg, a recombinase or helicase) to unwind an initial double stranded DNA molecule. Thus, also provided by the present invention is a method of amplifying a target DNA molecule in a sample, the method comprising:
a) providing a sample containing said target DNA molecule;
b) providing first and second DNA primers (P2p, P2*) complementary to contiguous portions of said target DNA molecule;
c) providing third and fourth DNA primers (P1c*, P1(csp)) complementary to the P2p and P2* primers, wherein the third and fourth DNA primers are destabilising primers;
d) providing a single-strand DNA binding protein (SSB) and a DNA unfolding enzyme;
e) allowing the SSB and DNA unfolding enzyme to separate the strands of the target DNA molecule, so as to allow the P2p and P2* primers to anneal to the target DNA, to form a DNA-nicked DNA duplex;
f) ligating the P2p and P2* primers, to form a DNA-DNA duplex, wherein one DNA strand is ligated P2p-P2*;
g) allowing the ligated P2p-P2* DNA strand to dissociate from the duplex;
h) allowing the destabilising primers to anneal to the ligated P2p-P2* DNA strand to form a DNA-nicked DNA duplex;
i) ligating the P1c* and P1(csp) primers, to form a product DNA duplex having a first cDNA strand corresponding to the P2p-P2* primers, and a second destabilised cDNA strand corresponding to the destabilising P1c*-P1(csp) primers;
j) allowing the destabilised cDNA strand to dissociate from the first cDNA strand;
k) allowing the P2p and P2* primers to anneal to the destabilised cDNA strand, and allowing the destabilising primers to anneal to the first cDNA strand to form DNA-nicked DNA duplexes;
l) ligating the primers to form product DNA duplexes having a first cDNA strand corresponding to the P2p-P2* primers, and a second destabilised cDNA strand corresponding to the destabilising P1c*-P1(csp) primers; and
m) repeating steps j) to l) to produce multiple copies of the first and second cDNA strands;
   wherein the ligating steps are carried out with a DNA ligase having no single base overhang or blunt end ligating ability.

The SSB may be a bacterial SSB. The DNA unfolding enzyme may be a DNA helicase, or may be a DNA recombinase, preferably RecA.

The DNA ligase is preferably PBCV-1 DNA ligase. Other features of this aspect of the invention may be the same as for the RNA amplification method described herein. It will be noted that no displacement DNA is required in this method, as the ligated DNA will spontaneously dissociate from the template once formed. Further, we believe that this method—using SSB and helicase or recombinase—can also serve to replace the disDNA from the RT-LIDA method described herein. As such, a further aspect of the invention provides a method of amplifying a target RNA molecule in a sample, the method comprising:
a) providing a sample containing said target RNA molecule;
b) providing first and second DNA primers (P2p, P2*) complementary to contiguous portions of said target RNA molecule;
c) providing third and fourth DNA primers (P1c*, P1(csp)) complementary to the P2p and P2* primers, wherein the third and fourth DNA primers are destabilising primers;
d) providing a single-strand DNA binding protein (SSB) and a DNA unfolding enzyme;
e) allowing the P2p and P2* primers to anneal to the target RNA, to form a RNA-nicked DNA duplex;
f) ligating the P2p and P2* primers, to form an RNA-DNA duplex, wherein the DNA strand is ligated P2p-P2*;
g) allowing the SSB and DNA unfolding enzyme to displace the ligated P2p-P2* DNA strand from the duplex;
h) allowing the destabilising primers to anneal to the ligated P2p-P2* DNA strand to form a DNA-nicked DNA duplex;
i) ligating the P1c* and P1(csp) primers, to form a product DNA duplex having a first cDNA strand corresponding to the P2p-P2* primers, and a second destabilised cDNA strand corresponding to the destabilising P1c*-P1(csp) primers;
j) allowing the destabilised cDNA strand to dissociate from the first cDNA strand;
k) allowing the P2p and P2* primers to anneal to the destabilised cDNA strand, and allowing the destabilising primers to anneal to the first cDNA strand to form DNA-nicked DNA duplexes;
l) ligating the primers to form product DNA duplexes having a first cDNA strand corresponding to the P2p-P2* primers, and a second destabilised cDNA strand corresponding to the destabilising P1c*-P1(csp) primers; and
m) repeating steps j) to l) to produce multiple copies of the first and second cDNA strands;
wherein the ligating steps are carried out with a DNA ligase having no single base overhang or blunt end ligating ability.

Also provided is a kit comprising oligonucleotides having the sequences of SEQ ID NOs: 3-7, and optionally also SEQ ID NOs: 8 and 9. The oligonucleotide having SEQ ID NO: 8 may be provided immobilised on a solid support, and with the oligonucleotide of SEQ ID NO: 9 hybridised thereto.

The present inventor has also found that the reaction mix can be separated into a liquid master mix and a lyophilised reagent component is possible. The invention therefore further provides a kit for amplification of a target RNA sequence, the kit comprising:
a) a liquid master mix comprising PBCV-1 DNA ligase, Tris, MgCl₂, ATP, and DTT;
b) in lyophilised form, oligonucleotide primers and displacement DNA suitable for performing RT-LIDA as described herein.

The kit may further comprise a solid support having a reporter oligonucleotide immobilised thereon, and a quencher oligonucleotide hybridised to the reporter oligonucleotide.

The master mix may comprise 1.05 UM DNA ligase, 50 mM Tris, 10 mM MgCl₂, 1 mM ATP and 10 mM DTT. In embodiments, the master mix and/or the lyophilised components may comprise a crowding agent, preferably PEG.

In embodiments, the solid support is in the form of a reaction vessel, where the reaction may take place. The lyophilised reagents may be provided in the reaction vessel. In this way, a sample containing RNA to be detected and the master mix may be added to the reaction vessel containing the lyophilised reagents, and the whole amplification and detection process take place in the vessel.

A yet further aspect of the invention relates to an alternative liquid master mix. In this embodiment, the master mix comprises Tris, manganese cations, DTT, and below 1 mM ATP, at pH greater than 8. A particularly preferred master mix comprises 50 mM Tris-HCl, 5 mM MnCl₂, 10 μM ATP, 10 mM DTT, at pH 8.5. This differs from the conventional master mix for PBCV-1, which contains higher ATP concentrations, has a lower pH, and contains MgCl₂ rather than MnCl₂. See for example the product data sheet for SplintR ligase available from New England Biolabs. As explained further herein, this modified master mix reduces the production of adenylylated DNA, a byproduct which can reduce production of the correct ligated product. The mix may further comprise PBCV-1 DNA ligase, preferably at 1.05 UM, and/or may further comprise a crowding agent, preferably PEG. This master mix may also be provided together with lyophilised reagents as described.

### BRIEF DESCRIPTION OF THE DRAWINGS

FIG. 1 shows a genome map of SARS-CoV-2

FIG. 2. SARS-CoV-2 ORF9c. Red shaded sequences represent the similarity between the protein sequences and the location of the 3 amino acid ‘LTD’ terminal insertion is marked by arrows

FIG. 3. Nucleotide sequence of a portion of SARS-CoV-2 ORF9c, and location of relevant oligonucleotides.

FIG. 4. Overview of RT-LIDA process.

FIG. 5. SARS-CoV-2 ORF9c RT-LIDA oligonucleotides.

FIG. 6. Formation of amplified product with T4 ligase

FIG. 7. Comparison of single base overhang ligation by T4 and PBCV-1 ligases.

FIG. 8. Use of PCBV-1 ligase in positive and negative control samples.

FIG. 9. Kinetics of DNA ligation on RNA template.

FIG. 10. Displacement reporting strategy.

FIG. 11. Oligonucleotide sequences used in reporting strategy

FIG. 12. Indicator of test results.

FIG. 13. Example of test results.

FIG. 14. Modified displacement reporting strategy.

### DETAILED DESCRIPTION OF THE INVENTION

Coronaviruses (CoVs) (order Nidovirales, family Coronaviridae, subfamily Coronavirinae) are enveloped viruses with a positive sense, single-stranded RNA genome. With genome sizes ranging from 26 to 32 kilobases (kb) in length. They infect humans and cause disease to varying degrees, from upper respiratory tract infections (URTIs) resembling the common cold, to lower respiratory tract infections (LRTIs) such as bronchitis, pneumonia, and even severe acute respiratory syndrome (SARS). SARS-CoV-2, SARS-COV and MERS-COV cause severe infections that lead to high mortality rates.

The coronaviral genome encodes four major structural proteins: the spike (S) protein, nucleocapsid (N) protein, membrane (M) protein, and the envelope (E) protein, all of which are required to produce a structurally complete viral particle. The E protein is the smallest of the major structural proteins. During the replication cycle, E is abundantly expressed inside the infected cell, but only a small portion is incorporated into the virion envelope. E participates in viral assembly, release of virions and pathogenesis of the virus. Much of the protein is localised at the site of intracellular trafficking, the ER, Golgi, and ERGIC, where it participates in CoV assembly and budding.

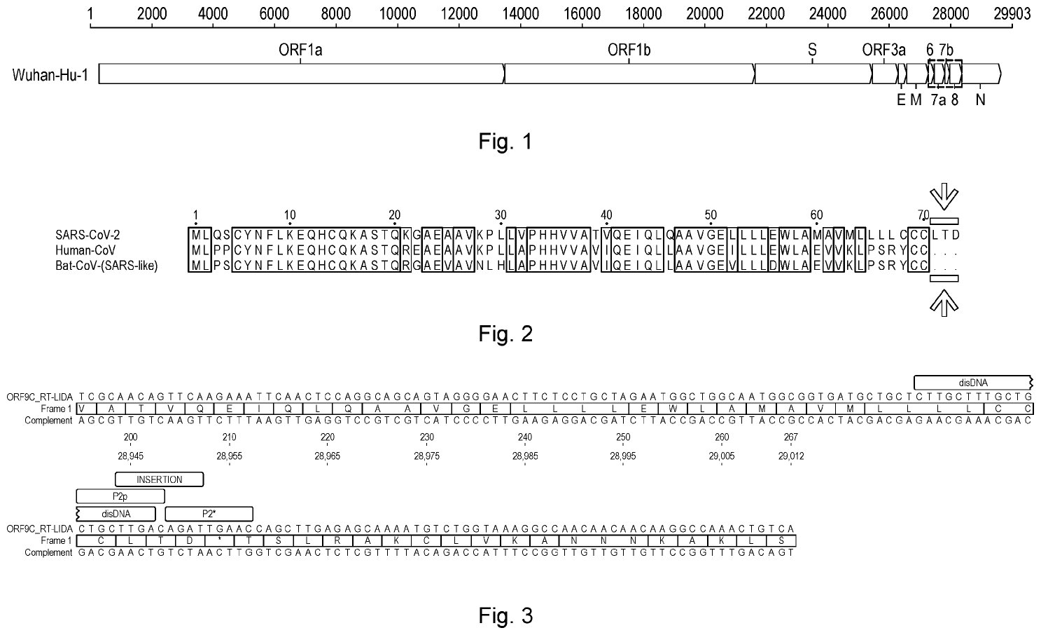

Sequenced genomes of SARS-CoV-2 strains combined with comparative analysis of the SARS-COV genome organization and transcription has been used to construct a tentative list of gene products. It was suggested that SARS-CoV-2 had 16 predicted non-structural proteins constituting a polyprotein (wORF1ab), followed by (at least) 13 downstream open reading frames (ORFs): Surface glycoprotein (or Spike), ORF3a, ORF3b, Envelope, Membrane, ORF6, ORF7a, ORF7b, ORF8, Nucleocapsid, ORF9a, ORF9b and ORF10. The three viral species whose proteins shared the highest similarity were consistently the same: human SARS coronavirus (SARS-COV), bat coronavirus (BtCoV), as well as another bat betacoronavirus (BtRf-BetaCoV). A genome map of SARS-CoV-2 is shown in FIG. 1.

A growing number of mutations have already been identified in clinical samples of SARS-CoV-2. It is therefore important to design an assay to regions that are impacted least by mutations, or by selecting a chemistry that can report mutations back to the clinician or researcher.

A number of these mutations have been shown to span sites used in commercially available assays which have altered the performance of these tests. In some, multiple mutations across the primer, probe, or primer and probe sites have been observed.

Rather than design a set of primers and probes to a variable region in the spike protein, we selected a region that is not present in other SARS or coronaviruses and is relatively conserved in SARS-CoV-2. We have designed a test that spans an insertion unique to SARS-CoV-2 making it highly specific. An advantage of selecting this target is that testing for SARS-CoV-2 can be carried out using a single target; other assays for SARS-CoV-2 typically require use of at least two separate genomic targets due to the potential for cross reactivity with other coronaviruses from non-unique targets.

Specifically, we have designed a RT-LIDA assay targeted to ORF9c (previously known as ORF14). This is a 70 amino acid protein which was previously of unknown function and present in Human SARS and Bat CoV. In SARS-CoV-2 the ORF9c protein is 73 amino acid long and has a 9 bp insert coding 3 additional amino acids at the terminus of the transcript (FIG. 2 shows a comparison of the ORF9c amino acid sequences from SARS-CoV-2 (SEQ ID NO: 12), Human SARS (SEQ ID NO: 13), and Bat CoV (SEQ ID NO: 14)). More recently this protein has been shown to play a critical and important role in the viruses' ability to evade the human immune system; given the importance of this protein, and the observed lack of variability in the ORF9c sequence, this highly-conserved region appears very suitable as a diagnostic target.

FIG. 3 shows the nucleotide sequence of cDNA obtained from the SARS-CoV-2 genome spanning the ORF9c insert (SEQ ID NO: 15), along with the amino acid sequence (SEQ ID NO: 16) and the complementary DNA sequence (SEQ ID NO: 17). Marked on the figure are the locations from which the displacement DNA (disDNA), and first and second primers (P2p, P2*) are derived.

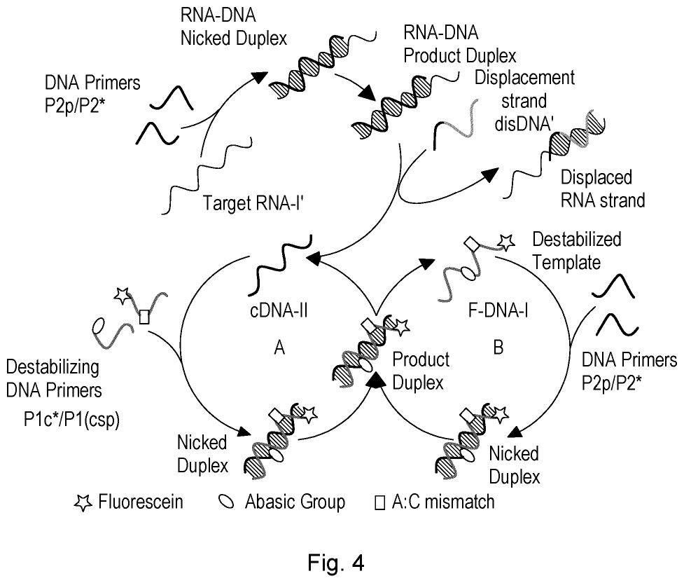

The overall process of RT-LIDA is shown in FIG. 4. In the upper panel, RNA-triggered ligation takes place, as linear amplification, to generate a cDNA from a template. In the lower panel, the cDNA is then amplified exponentially. A sample containing a target RNA (“Target RNA-I”) is combined with DNA primers P2p and P2*, which hybridise to adjacent portions of Target RNA-I′ to form an RNA-nicked DNA duplex. Ligase in the reaction mix then repairs the nick, resulting in an RNA-DNA duplex. A displacement DNA strand (disDNA′) which partially overlaps the cDNA preferentially hybridises with the RNA, displacing the generated cDNA-II strand.

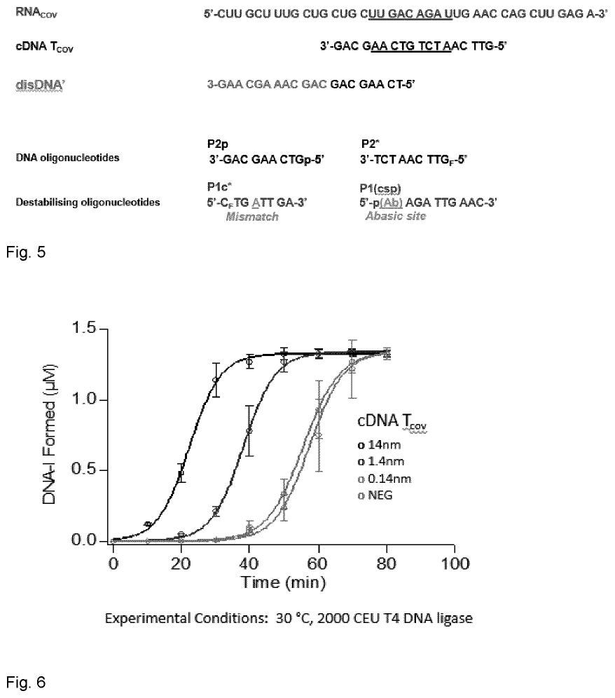

The reaction then moves into the exponential phase, in which the DNA primers P2p and P2* and the destabilising DNA primers P1c* and P1(csp) alternately hybridise to the c-DNA-II or F-DNA-I strands, are ligated, and then spontaneously dissociate from the hybridised strand due to the presence of the destabilising features of an abasic site and an internal mismatch in the destabilising primers. Each cycle thus doubles the number of cDNA strands. In this illustration, the destabilising primers include a fluorescent label, allowing detection of the F-DNA-I strand after formation. FIG. 5 shows the various oligonucleotides used in the RT-LIDA detection method for SARS-CoV-2. RNAcov (SEQ ID NO: 10) is a synthetic RNA template used in assay testing.

The conventional RT-LIDA process uses T4 ligase as the ligation enzyme. However, as shown in FIG. 6, this enzyme results in false positives after a certain period of time. Specifically, FIG. 6 shows production of DNA-I product at different starting concentrations of template cDNA. Even with a negative sample, DNA-I is still produced. This is thought to be thanks to formation of oligonucleotide primer duplexes in the reaction mix-T4 ligase will spontaneously ligate such duplexes if there is a single base overhang, and such ligated oligonucleotides will seed amplification as the reaction progresses. Clearly, this is undesirable.

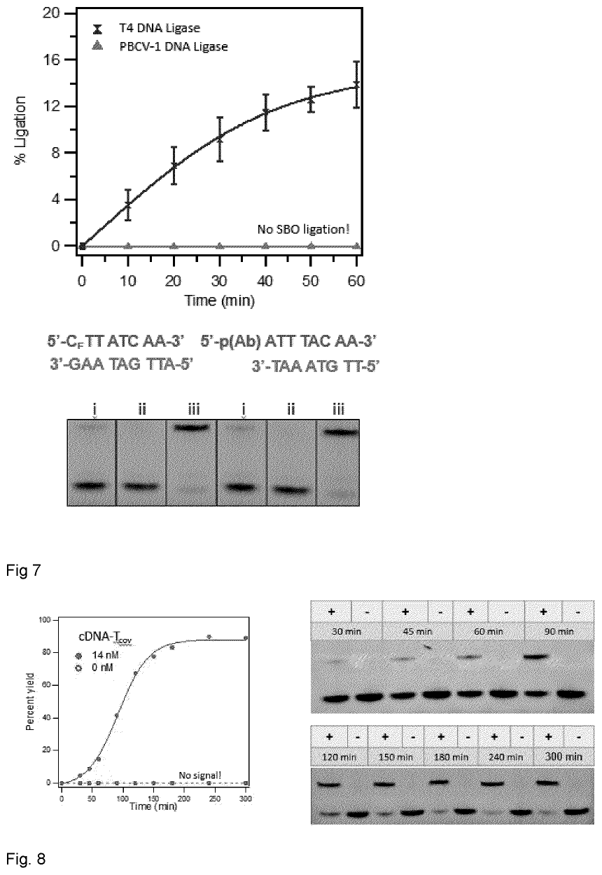

We therefore investigated the use of alternative ligases lacking single base overhang ligation ability. PBCV-1 ligase is described in Nucleic Acids Research, 2003, Vol. 31, No. 17 DOI: 10.1093/nar/gkg665; and ligation of RNA-splinted DNA by PBCV-1 ligase is described in G Lohman et al, “Efficient DNA ligation in DNA-RNA hybrid helices by Chlorella virus DNA ligase”; Nucleic acids research, 42(3), 1831-1844. https://doi.org/10.1093/nar/gkt1032. FIG. 7 compares ligation of single base overhangs by T4 and PBCV-1 ligases. There is no ligation with the PBCV-1 ligase. The lower portion of the figure shows gel electrophoresis of the products. Low-level ligation of single base overhang with 5′-abasic phosphate is shown to occur with T4 DNA Ligase (i). No SBO ligation was detectable with PBCV-1 DNA Ligase (ii). For comparative purpose full ligation product initiated with 14 nM target is shown (iii). No ligation of the 5′-abasic phosphate with PBCV-1 DNA Ligase avoids background amplification observed with T4 DNA ligase.

The results of using PBCV-1 ligase in positive and negative control samples are shown in FIG. 8. Percent yield is based on ligation of fluorescent labeled P1c oligonucleotide. Enzyme was added directly to the amplification mixture. It can be seen that no signal was observed in the negative control, even after 300 minutes.

Under recommended conditions, PBCV-1 reaction buffer contains 50 mM Tris-HCl, 10 mM MgCl₂, 1 mM ATP, 10 mM DTT, and the reaction is carried out at pH 7.5 at 25° C. However, this can result in generation of adenylylated DNA primers which reduce yield of the correct ligation product. PBCV-1 DNA ligase binds to a nicked DNA duplex containing reactive 3′-OH and 5′-PO₄ termini. It does not bind to a continuous DNA duplex, to a tailed duplex or even to a nicked ligand containing non-ligatable 3′-OH and 5′-OH termini.

The ATP-dependent DNA ligases catalyse the joining of 5′-phosphate-terminated strands to 3′-hydroxyl-terminated strands via three sequential nucleotidyl transfer reactions. In the first step, attack on the α-phosphate of ATP by DNA ligase results in displacement of pyrophosphate and formation of a covalent ligase-adenylate intermediate in which AMP is linked to the ε-amino group of a lysine. The active site lysine residue is located within a conserved motif, KxDGxR. The AMP is then transferred to the 5′-monophosphate terminus of a nicked DNA duplex to form the DNA-adenylate intermediate, which consists of an inverted (5′)-(5′) pyrophosphate bridge structure, AppDNA. Attack by the 3′-OH-terminated strand of the nicked duplex on DNA-adenylate seals the nick and releases AMP.

If AppDNA is released in solution, it can become a ‘dead end’ product under conditions of mM ATP concentrations, as free ligase rapidly reacts with ATP to adenylylate the active site of the enzyme. The adenylylated enzyme cannot bind AppDNA, as the adenylyl group on the enzyme occupies the same binding pocket as the adenylyl group on the AppDNA intermediate. UM ATP concentrations result in a higher steady state concentration of deadenylylated ligase, which can bind and react AppDNA substrates to ligated DNA effectively.

As well as using μM ATP (e.g. 10 μM), enzyme concentration and selection of Mn2+ (5 mM) instead of Mg2+ significantly reduces the formation of AppDNA and the potential for dead-end substrates. pH 8.5 also eliminates AppDNA, normally pH between 7 and 8 is used for most ligase master mixes. Hence, the invention further provides a ligase buffer comprising manganese cations, a reduced (less than 1 mM) amount of ATP, and at a pH above 8. A preferred ligase buffer for use with the methods of the invention includes 50 mM Tris-HCl, 5 mM MnCl₂, 10 UM ATP, 10 mM DTT, at pH 8.5.

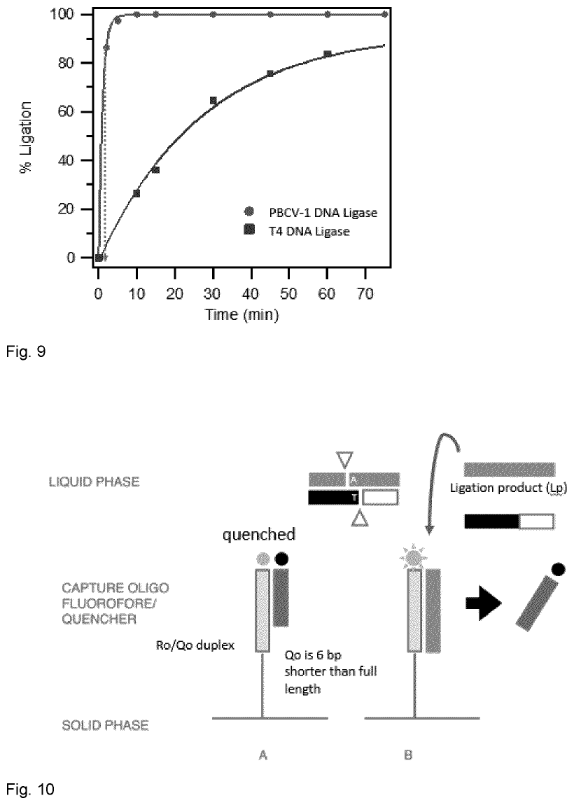

Further investigation demonstrated that PBCV-1 ligase additionally ligates DNA primers on an RNA template more rapidly than T4 ligase. See FIG. 9. Here the RNA template was 5′-CUU GCU UUG CUG CUG CUU GAC AGA UUG AAC CAG CUU GAG A-3′ (“RNAcov”; SEQ ID NO: 10), and the P2p and P2* primers used. The more rapid ligation with PBCV-1 has benefits in reducing the time taken for the RNA-templated step but critically may also be important in allowing the displacement DNA to initiate LIDA after the RNA-template step by optimizing the kinetics of both reactions. Specifically, reducing the length of the disDNA will increase the time taken for displacement of the RT-ligation product. Use of PBCV-1 therefore permits the ligation to occur prior to displacement, to ensure generation of the initial cDNA product. This improves sensitivity, and reduces the chance of false negatives. Reactions were carried out with PBCV-1 DNA Ligase 1.05 UM, 50 mM Tris 10 mM MgCl2, 1 mM ATP and DTT; or with T4 DNA Ligase 2000 CEU, 50 mM Tris 10 mM MgCl2, 10 μM ATP. Further, these reaction kinetics also permit the use of molecular crowding agents, such as PEG, in the reaction mix with PBCV-1 to further accelerate the reaction.

An illustration of the reporting strategy is shown in FIG. 10. The ligation and amplification steps are carried out in a liquid phase, in contact with a solid support on which is immobilised a reporter oligonucleotide Ro including a reporter dye. This is initially hybridised to a shorter complementary oligonucleotide Qo which includes a quencher molecule. Importantly, the Ro oligonucleotide is the same length as, and fully complementary to, one of the cDNA product strands; while the Qo oligonucleotide has the same sequence as a part of the cDNA product strand but is shorter than full length (here, 6 nt shorter), and longer than either of the individual primers. The cDNA, if present, will therefore displace the Qo oligonucleotide, separating the reporter and quencher, and allowing detection of the reporter. The smaller individual primers cannot displace the Qo oligonucleotide, so the system is not prone to false positives. Further, the released Qo oligonucleotide cannot take part in ligation reactions effected by PBCV-1 if the overhang is too short (for example, 3 nt, where the whole Qo is 6 nt shorter than the cDNA) to allow nick ligation; again, this reduces the chance of false positives. The relevant sequences are illustrated in FIG. 11 (Ro, SEQ ID NO: 8; Qo, SEQ ID NO: 9; LIDA ligation product, SEQ ID NO: 11. LIDA ligation product is P1c*, SEQ ID NO: 6, and P1(csp), SEQ ID NO: 7).

In other embodiments, the primers may also include an additional sequence tag which is not part of the target region to be amplified; this allows use of a reporter sequence which is in part complementary to the sequence tag, and does not require any sequence homology to the priming regions as such. This reduces the risk of sequences binding to the reporter or released sequences. As an example, if the sequence to be detected is the P2p-P2* ligated oligonucleotide, then the P2* primer may include an additional sequence tag: P2p-P2*-T. The reporter oligonucleotide Ro is complementary to the P2p-P2*-T sequence as a whole, whereas the quencher oligonucleotide Qo omits the tag, so would have the P2p-P2* sequence. In this embodiment, the released Qo oligonucleotide is able to serve as a template for the P1 primers; however, release only takes place as the P2p-P2*-T product accumulates, such that signal is only detected when there is genuine amplification and release. This modified displacement reporting strategy is illustrated in FIG. 14.

A particular advantage of this combination with the described removal of false positive product formation, is that the amplification always goes to completion (100%) after a certain time regardless of input target RNA concentration, so that there is minimal requirement to evaluate fluorescence strength as an endpoint determination, it provides a yes/no result which is particularly suited to POC and OTC applications. However, monitoring of fluorescence signal as a function of time can be used to provide a quantitative measurement for professional use where determination of the quantity of RNA in the sample is important. This method can be used in both modalities.

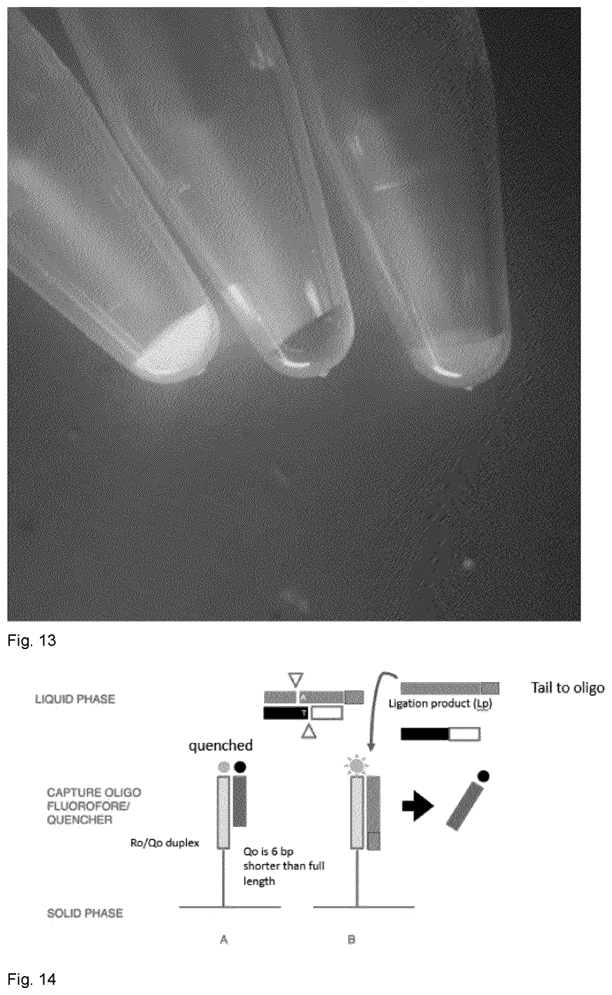

FIG. 13 shows a proof of concept of the assay and reporting strategy described herein. Three assay tests are shown; from left to right, these are unquenched Ro reporter oligo; quenched signal from a Ro/Qo reporter-quencher pair; and positive signal after displacement of the quencher oligonucleotide from the reporter by addition of a SARS-CoV-2 ligation product.

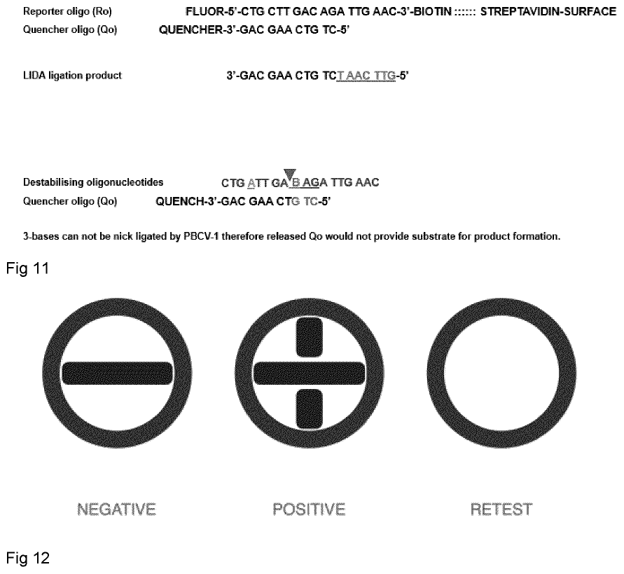

Finally, an illustration of how the reporting strategy may appear is shown in FIG. 12. Reporter-quencher oligos may be laid out in a cross shape, with one arm of the cross being a reporter for the positive test (eg, SARS-CoV-2), and the other being a reporter for a control incorporated in the test (eg, a human mRNA expected to be present in the sample). Development of the reporter therefore provides a simple indication of whether the test is negative or positive.

In summary, then, we have developed an assay for SARS-CoV-2 which identifies a highly conserved region which is distinct from closely related viruses. Further, in the process of development we have determined that use of PBCV-1 ligase in an RT-LIDA reaction has a number of benefits:
unlike T4 DNA ligase it does not ligate a single overhang with the 5′-abasic phosphate which completely eliminates the background triggered ligation observed with T4 DNA ligase;
PBCV-1 DNA Ligase catalyzes RNA-templated ligation of DNA fragments much faster than T4 DNA ligase; this shortens the time for this step to a couple of minutes;
optimizing the length of the disDNA oligonucleotide could allow RT to happen before displacement activity removes the DNA oligos from the RNA which normally would limit the efficiency of this critical ligation step. This would allow a single step process.

Further, adoption of the displacement reporting method would be particularly useful for LIDA and in fact is based on the same displacement mechanics of oligonucleotide association disassociation kinetics within the LIDA assay. This too can be carried out as a single step process.

We have additionally developed an improved ligation master mix, the use of which may reduce production of AppDNA during ligation, which can reduce yield of the correct ligation product.

Further, use of SSB and DNA unwinding proteins, such as RecA or helicase, can allow a single step amplification procedure to be carried out on DNA, as well as RNA.

These properties mean that the assays described herein may be carried out rapidly, in a single reaction vessel, and in a single step.

SEQUENCE LISTING

3′-AAC TGT CTA-5′, SEQ ID NO: 1-cDNA sequence of LTD insertion
5′-UUG ACA GAU-3′, SEQ ID NO: 2-genomic sequence of LTD insertion
3′-GAA CGA AAC GAC GAC GAA CT-5′, SEQ ID NO: 3-disDNA sequence
3′-GAC GAA CTGp-5′, SEQ ID NO: 4-P2p seq
3′-TCT AAC TTG*- 5′, SEQ ID NO: 5-P2* seq, *is label
5′-C*TG ATT GA-3′, SEQ ID NO: 6-P1c* seq
5′-p(Ab) AGA TTG AAC-3′, SEQ ID NO: 7-P1(csp) seq,
(Ab) is abasic site
5′-CTG CTT GAC AGA TTG AAC-3′ SEQ ID NO: 8, reporter oligo
3′-GAC GAA CTG TC-5′, SEQ ID NO: 9, quencher oligo
5′-CUU GCU UUG CUG CUG CUU GAC AGA UUG AAC CAG CUU GAG A-3′
SEQ ID NO: 10, RNAcov
3′-GAC GAA CTG TCT AAC TTG-5′, SEQ ID NO: 11, LIDA ligation product
SEQ ID NO: 12-SARS-COV-2 sequence from FIG. 2
SEQ ID NO: 13-Human-CoV sequence from FIG. 2
SEQ ID NO: 14-Bat-CoV (SARS-like) sequence from FIG. 2
SEQ ID NO: 15-ORF9c_RT-LIDA sequence from FIG. 3
SEQ ID NO: 16-frame 1 sequence from FIG. 3
SEQ ID NO: 17-complement of SEQ ID NO: 15

1. A method for detecting SARS-CoV-2 in a sample, the method comprising: generating cDNA from an RNA present in the sample; amplifying a portion of the cDNA using an amplification process specific for a portion of the cDNA corresponding to the SARS-CoV-2 genome coding for ORF9c; and detecting the presence of a portion of amplified cDNA coding for a Leu-Thr-Asp (LTD) sequence at or near the terminus of the ORF9c protein.

2. The method of claim 1 wherein amplification is via RT-LIDA.

3. The method of claim 2, wherein the RT-LIDA comprises use of a DNA ligase having no single base overhang or blunt end ligating ability; preferably wherein the DNA ligase is PBCV-1 DNA ligase.

4. The method of any preceding claim wherein the detection step comprises capturing at least one of the cDNA strands via a complementary oligonucleotide, optionally immobilised on a solid support.

5. The method of claim 4 wherein the immobilised complementary oligonucleotide is initially hybridised to a partially-complementary oligonucleotide; and capturing the cDNA strand comprises allowing the cDNA strand to displace the partially-complementary oligonucleotide.

6. The method of claim 5 wherein the partially-complementary oligonucleotide is shorter than the immobilised oligonucleotide and is shorter than the cDNA.

7. The method of claim 5 or 6 wherein the immobilised complementary oligonucleotide and partially-complementary oligonucleotide include a reporter-quencher pair.

8. The method of any of claims 5 to 7 wherein the displaced partially-complementary oligonucleotide does not form a substrate for further amplification.

9. The method of any preceding claim wherein the amplification comprises use of primers having nucleotide sequences of SED ID NOs: 4-7.

10. An assay for SARS-CoV-2 wherein a portion of a nucleic acid coding for a Leu-Thr-Asp (LTD) sequence at or near the terminus of the ORF9c protein is amplified and detected.

11. A kit comprising oligonucleotides having the sequences of SEQ ID NOs: 3-7, and optionally also SEQ ID NOs: 8 and 9.

12. The kit of claim 11 wherein the oligonucleotide having SEQ ID NO: 8 is immobilised on a solid support.

13. A method of amplifying a target RNA molecule in a sample, the method comprising:
a) providing a sample containing said target RNA molecule;
b) providing first and second DNA primers (P2p, P2*) complementary to contiguous portions of said target RNA molecule;
c) providing third and fourth DNA primers (P1c*, P1(csp)) complementary to the P2p and P2* primers, wherein the third and fourth DNA primers are destabilising primers;
d) providing a displacement DNA strand (disDNA) overlapping with the P2p and or the P2* primers and complementary to the target RNA molecule;
e) allowing the P2p and P2* primers to anneal to the target RNA, to form a RNA-nicked DNA duplex;
f) ligating the P2p and P2* primers, to form an RNA-DNA duplex, wherein the DNA strand is ligated P2p-P2*;
g) allowing the disDNA to displace the ligated P2p-P2* DNA strand from the duplex;
h) allowing the destabilising primers to anneal to the ligated P2p-P2* DNA strand to form a DNA-nicked DNA duplex;
i) ligating the P1c* and P1(csp) primers, to form a product DNA duplex having a first cDNA strand corresponding to the P2p-P2* primers, and a second destabilised cDNA strand corresponding to the destabilising P1c*-P1(csp) primers;
j) allowing the destabilised cDNA strand to dissociate from the first cDNA strand;
k) allowing the P2p and P2* primers to anneal to the destabilised cDNA strand, and allowing the destabilising primers to anneal to the first cDNA strand to form DNA-nicked DNA duplexes;
l) ligating the primers to form product DNA duplexes having a first cDNA strand corresponding to the P2p-P2* primers, and a second destabilised cDNA strand corresponding to the destabilising P1c*-P1(csp) primers; and
m) repeating steps j) to l) to produce multiple copies of the first and second cDNA strands;
wherein the ligating steps are carried out with a DNA ligase having no single base overhang or blunt end ligating ability.

14. The method of claim 13 wherein the destabilising primers include one or more features selected from the presence of an abasic site or a mismatch with the corresponding complementary sequence.

15. The method of claim 14 wherein one destabilising primer includes a mismatch, and one destabilising primer includes an abasic site.

16. The method of any of claims 13-15 wherein the upstream primer of each pair includes a phosphate group at the 5′ end.

17. The method of any of claims 13-16 further comprising the step of detecting at least one of the cDNA strands.

18. The method of any of claims 13-17 wherein at least one of the primers includes a label.

19. The method of claim 17 or 18 wherein the detection step comprises capturing at least one of the cDNA strands via a complementary oligonucleotide, optionally immobilised on a solid support.

20. The method of claim 19 wherein the immobilised complementary oligonucleotide is initially hybridised to a partially-complementary oligonucleotide; and capturing the cDNA strand comprises allowing the cDNA strand to displace the partially-complementary oligonucleotide.

21. The method of claim 20 wherein the partially-complementary oligonucleotide is shorter than the immobilised oligonucleotide and is shorter than the cDNA.

22. The method of claim 20 or 21 wherein the immobilised complementary oligonucleotide and partially-complementary oligonucleotide include a reporter-quencher pair.

23. The method of any of claims 20 to 22 wherein the displaced partially-complementary oligonucleotide does not form a substrate for further amplification.

24. The method of any of claims 13-23, wherein the target RNA molecule is viral RNA, preferably SARS-CoV-2 RNA.

25. The method of any of claims 13-24 wherein the primers have nucleotide sequences of SED ID NOs: 4-7.

26. The method of any of claims 13-25 wherein the DNA ligase is PBCV-1 DNA ligase.

27. A kit for amplification of a target RNA sequence, the kit comprising:
a) a liquid master mix comprising PBCV-1 DNA ligase, Tris, MgCl₂, ATP, and DTT;
b) in lyophilised form, oligonucleotide primers and displacement DNA suitable for performing RT-LIDA.

28. The kit of claim 27 further comprising a reporter oligonucleotide, and a quencher oligonucleotide hybridised to the reporter oligonucleotide, wherein optionally the reporter oligonucleotide is immobilised on a solid support.

29. A ligation buffer that limits the production of AppDNA ligation intermediary complex through selection of one or more of pH>=8.0, ATP<=1 mM, and use of manganese cations.

30. A method of amplifying a target DNA molecule in a sample, the method comprising:
a) providing a sample containing said target DNA molecule;
b) providing first and second DNA primers (P2p, P2*) complementary to contiguous portions of said target DNA molecule;
c) providing third and fourth DNA primers (P1c*, P1(csp)) complementary to the P2p and P2* primers, wherein the third and fourth DNA primers are destabilising primers;
d) providing a single-strand DNA binding protein (SSB) and a DNA unfolding enzyme;
e) allowing the SSB and DNA unfolding enzyme to separate the strands of the target DNA molecule, so as to allow the P2p and P2* primers to anneal to the target DNA, to form a DNA-nicked DNA duplex;
f) ligating the P2p and P2* primers, to form a DNA-DNA duplex, wherein one DNA strand is ligated P2p-P2*;
g) allowing the ligated P2p-P2* DNA strand to dissociate from the duplex;
h) allowing the destabilising primers to anneal to the ligated P2p-P2* DNA strand to form a DNA-nicked DNA duplex;
i) ligating the P1c* and P1(csp) primers, to form a product DNA duplex having a first cDNA strand corresponding to the P2p-P2* primers, and a second destabilised cDNA strand corresponding to the destabilising P1c*-P1(csp) primers;
j) allowing the destabilised cDNA strand to dissociate from the first cDNA strand;
k) allowing the P2p and P2* primers to anneal to the destabilised cDNA strand, and allowing the destabilising primers to anneal to the first cDNA strand to form DNA-nicked DNA duplexes;
l) ligating the primers to form product DNA duplexes having a first cDNA strand corresponding to the P2p-P2* primers, and a second destabilised cDNA strand corresponding to the destabilising P1c*-P1(csp) primers; and
m) repeating steps j) to l) to produce multiple copies of the first and second cDNA strands;
wherein the ligating steps are carried out with a DNA ligase having no single base overhang or blunt end ligating ability.

31. A method of amplifying a target RNA molecule in a sample, the method comprising:
a) providing a sample containing said target RNA molecule;
b) providing first and second DNA primers (P2p, P2*) complementary to contiguous portions of said target RNA molecule;
c) providing third and fourth DNA primers (P1c*, P1(csp)) complementary to the P2p and P2* primers, wherein the third and fourth DNA primers are destabilising primers;
d) providing a single-strand DNA binding protein (SSB) and a DNA unfolding enzyme;
e) allowing the P2p and P2* primers to anneal to the target RNA, to form a RNA-nicked DNA duplex;
f) ligating the P2p and P2* primers, to form an RNA-DNA duplex, wherein the DNA strand is ligated P2p-P2*;
g) allowing the SSB and DNA unfolding enzyme to displace the ligated P2p-P2* DNA strand from the duplex;
h) allowing the destabilising primers to anneal to the ligated P2p-P2* DNA strand to form a DNA-nicked DNA duplex;
i) ligating the P1c* and P1(csp) primers, to form a product DNA duplex having a first cDNA strand corresponding to the P2p-P2* primers, and a second destabilised cDNA strand corresponding to the destabilising P1c*-P1(csp) primers;
j) allowing the destabilised cDNA strand to dissociate from the first cDNA strand;
k) allowing the P2p and P2* primers to anneal to the destabilised cDNA strand, and allowing the destabilising primers to anneal to the first cDNA strand to form DNA-nicked DNA duplexes;
l) ligating the primers to form product DNA duplexes having a first cDNA strand corresponding to the P2p-P2* primers, and a second destabilised cDNA strand corresponding to the destabilising P1c*-P1(csp) primers; and
m) repeating steps j) to l) to produce multiple copies of the first and second cDNA strands;
wherein the ligating steps are carried out with a DNA ligase having no single base overhang or blunt end ligating ability.
